(19) United States
(12) Patent Application Publication
(10) Pub. No.: US 2004/0193023 A1
(43) Pub. Date: Sep. 30, 2004

(54) System, method and apparatus for monitoring recording and reporting physiological data

(76) Inventor: Aris Mardirossian, Germantown, MD (US)

Correspondence Address: NIXON & VANDERHYE, PC, 1100 N GLEBE ROAD, 8TH FLOOR, ARLINGTON, VA, 22201-4714, US

(21) Appl. No.: 10/401,139

(22) Filed: Mar. 28, 2003

(51) Int. Cl. A61B 5/00

(52) U.S. Cl. 600/309; 600/500; 600/505; 600/587; 600/365; 600/486; 600/407; 600/368; 600/476; 128/903

(57) ABSTRACT

A system, method and apparatus monitoring, recording and reporting physiological data includes an intravenous detection device in communication with at least one central processor. The intravenous detection device is configured to monitor at least one physiological measurement and report the measurement to the central processor via a communication medium. The central processor may store the data or immediately transmit the data to an authorized recipient. The central processor may also analyze the data to determine what further action should be taken, including immediate interventional action, such as, for example, defibrillation. The detection devices are preferably micro- or nano-level devices that are capable of flowing freely through the venous system of a host. The detection devices include a suitable transducer, an optional memory and a transceiver, and are preferably introduced into a host by injection of a fluid including a suspension of the detection devices. Measurements to be taken by the detection devices may include at least one of effective blood vessel diameter, fluid pressure, blood sugar level, cholesterol level, heart rate, heart rhythm, presence of a tumor, white blood cell count and visual image data.

### FIELD OF THE INVENTION

The present invention relates to a system, method and apparatus for monitoring various physiological conditions, storing data associated therewith and, if necessary, reporting the monitored condition in real-time to an appropriate authority. In particular, the invention is directed to a system employing microscopic sensors in the body to monitor, detect and record data with respect to various physiological conditions and to send this data to a central processor for further evaluation and transmission, as appropriate.

### BACKGROUND AND SUMMARY OF THE INVENTION

Tremendous advances in the fields of micro- and nano-technology have been realized recently, and advances in this field are continuing to occur at an almost exponential rate. Numerous developments have been made in the manufacture and application of so-called nanotechnology machines and circuits. For example, U.S. Pat. No. 6,510,359, to Merkle et al. describes systems and methods for manufacturing micro- and nano-level machines, the disclosure of which is hereby incorporated herein by reference. See also U.S. patent application Publication No. 2003/0012953, and U.S. Pat. No. 6,398,280, the disclosures of which are hereby incorporated herein by reference.

The ability to manufacture micro- and nano-level machines gives rise to tremendous applications of these technologies in, among other things, biological systems, such as, for example, the human body. Prior to the development of such small scale devices, it would be very cumbersome to implant devices into a living organism to measure physiological data. Instead, such measurements required tedious and time-consuming testing procedures using large scale, expensive equipment. Often times, such testing would require invasive procedures and result in extreme discomfort. Moreover, these types of measurements typically require a special visit to a hospital or doctor's office that require advance scheduling, thereby limiting their effectiveness in real-time determination of a problem.

The present invention overcomes the numerous deficiencies and drawbacks associated with such relatively large scale machines and the overhead expended in terms of human capital, wasted time and inefficient diagnosis, by taking advantage of the significant technological advances being made in the field of micro- and nano-technology.

In particular, exemplary embodiments of the present invention are directed to introducing micro- or nano-level devices into the living human body to measure various predetermined physiological conditions of the body in which the devices have been introduced. Because of the exceedingly small dimensions of these devices, they may be introduced into the body without substantial side-effects or discomfort. These devices may be introduced into, for example, the circulatory system of a host. Due to the small size of the devices, they can travel freely throughout the venous circulatory system of the body, taking various physiological measurements and observing physiological conditions of particular areas within the body.

The measurements and conditions observed by the devices may then be transmitted to one or more central processors located in or in close proximity to the host. The central processor, in turn, may evaluate the data received from the devices and determine what, if any, further action is to be taken. For example, the central processor may simply store the data for historical reference or later analysis by a physician. On the other hand, if the processor determines that the data support a diagnosis of a critical problem requiring immediate attention, for example, a blocked vessel, a blood clot, or the like, the central processor may act immediately to transmit this information to the appropriate authority for immediate action or closer monitoring. In addition, the central processor may determine that an abnormality requiring immediate action is detected. For example, the central processor may determine that detected heart rhythms indicate that the host is experiencing a cardiac arrhythmia requiring immediate defibrillation. The central processor may then, in conjunction with a pacemaker, cause delivery of a defibrillation pulse to either resume or sustain a normal cardiac rhythm until such time as a physician is available.

The central processor may also be configured to receive commands from an appropriate authority to alter the manner in which it stores or transmits data. For example, upon the detection of a condition that requires closer monitoring, for example, high blood sugar, high blood pressure, or the like, the processor may be commanded to continuously transmit the data in real-time to an appropriate authority, instead of merely storing data for later retrieval and analysis. Upon correction or alleviation of the particular condition, the central processor may be reprogrammed to resume its previous storage routines, for historical purposes, for example.

These and other features and advantages attendant with the present invention will be apparent to those skilled in the art from the following detailed description of exemplary embodiments of the invention made with reference to the following figures.

### BRIEF DESCRIPTION OF THE DRAWINGS

Exemplary embodiments of the invention will be described in detail herein with reference to the following drawings in which like reference numerals refer to like elements, and wherein:

FIG. 1 is an illustrative view of the human body showing the venous system and various devices according to an illustrative embodiment of the invention;

FIG. 2 is an illustrative block diagram according to an exemplary embodiment of the invention;

FIG. 3 is an illustrative block diagram of a measurement device according to an exemplary embodiment of the invention; and

FIG. 4 is a flowchart illustrating operation of an exemplary embodiment of the invention.

### DETAILED DESCRIPTION OF EXEMPLARY EMBODIMENTS

Exemplary embodiments of the invention will be described herein with reference to the figures. Operation of the exemplary embodiments of the invention will be described with reference to particular type of physiological measurement. However, it will be appreciated and understood that the exemplary embodiments of the invention apply equally to any number of different types of measurements and conditions as enumerated by example herein.

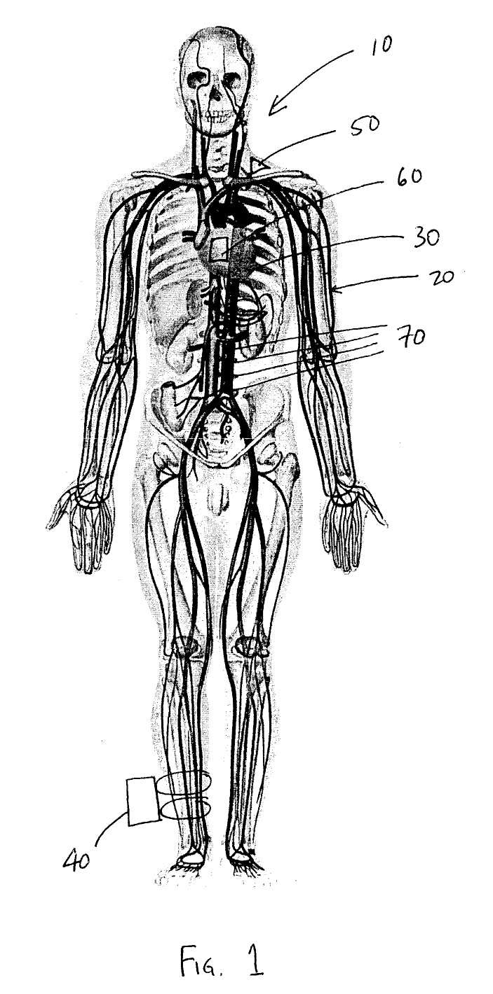

FIG. 1 is an illustrative view of the human body 10. It will be understood that the invention may apply equally to any living organism or to any mechanical device or machine in which monitoring is required. The body 10 includes a venous circulatory system 20 through which blood flows continuously, and is pumped by the heart 30.

According to an exemplary embodiment of the present invention, a plurality of micro- or nano-level devices 70 may be introduced into the venous system 20 of the body 10, using, for example, injection of a liquid suspension including the devices 70. The devices 70, thus introduced into the venous system 20, are free to circulate therethrough acquiring the various measurements which they are configured or programmed to make. The devices 70 (i.e., sensors) which freely circulate through the venous system of the body are typically not physically attached to any structure outside of the body, or to any structure which is manipulated by a doctor or the like.

Upon acquisition of these measurements, the devices 70 may either continuously or periodically transmit the measured data to a central processor 200 located in close proximity to, or within, the body 10. For example, a central processor may be co-located with a pacemaker 60, may be subcutaneously embedded in a predetermined area of the body 10, such as, for example, in the area of the shoulder as illustrated by reference numeral 50, or may be located on a device that is attached to, or in close proximity to, the body 10, such as, for example, strapped to the ankle as illustrated by reference numeral 40. It will be understood that the location of the central processor 200 will vary depending upon the condition of the body 10 and the particular application of the measurements being taken by the devices. It may also be advantageous to include multiple central processors in different locations to enable a determination of a particular device 70 within the body by, for example, measuring the differences in transmission time to each of the central processors 200. For example, it may be determined that a central processor 200 be located at the ankle 40 and the shoulder 50. By providing the ability to accurately determine the location of a particular device 70 the appropriate authority will better be able to determine which area of the body is producing a particular measurement, thereby enhancing the diagnosis and solving of a particular problem.

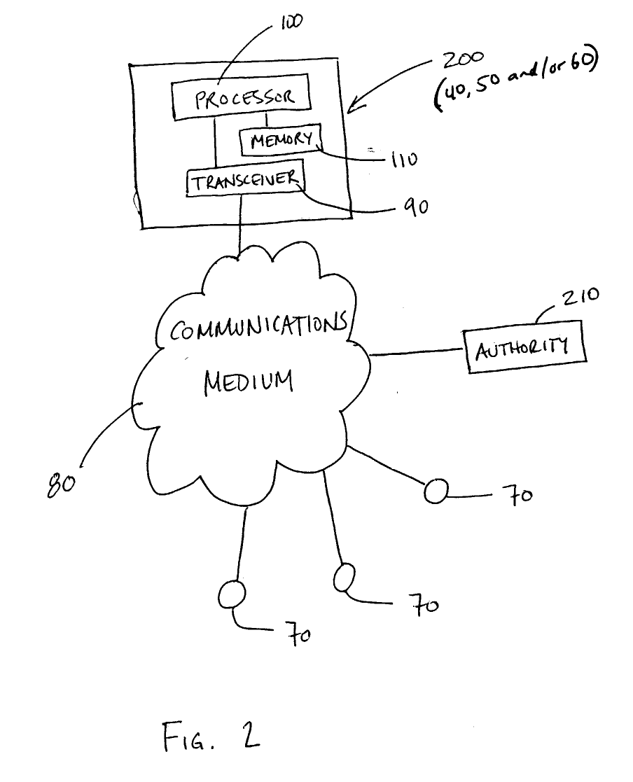

FIG. 2 is an illustrative block diagram of a central processor 200, including a communications medium 80 for enabling communication between the devices 70, the authorized authority 210 (e.g., a physician, hospital, or the like) and the central processor 200, according to an exemplary embodiment of the invention. The central processor 200 includes a processor 100 which may be, for example, optionally programmable, a memory 110 and a transceiver 90 for transmitting data from the central processor 200 to an appropriate authority 210, such as, for example, a physician or hospital. The transceiver 90 is also configured to receive data from the devices 70 via the communications medium 80, and may also receive re-programming data from an authorized source 210. The communications medium 80 may be, for example, a radio-frequency (RF) communications medium or the like.

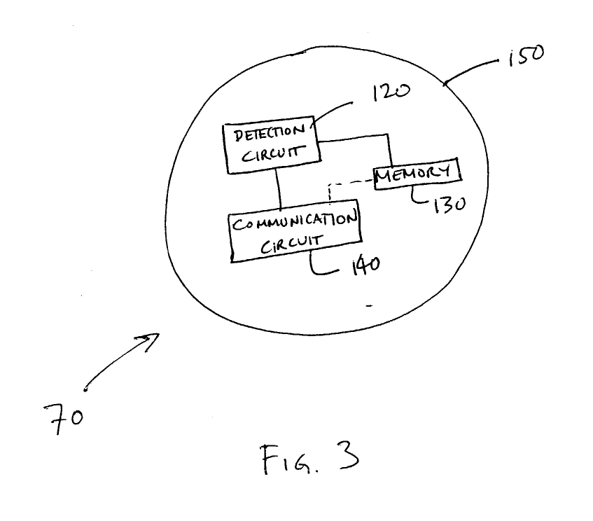

FIG. 3 is a block diagram illustrating various exemplary features of the micro- or nano-level devices 70. The devices 70 include detection circuitry 120, an optional memory circuit 130 and a transmitter circuit 140. The detection circuitry 120 is chosen based on the type of measurement being made. There are numerous different known transducers for measuring different types of physiological data. For example, the detection circuit 120 may be configured to monitor blood sugar levels. On the other hand, the detection circuit 120 may be configured to make ultrasonic measurements of various vascular conditions. Additionally, the detection circuit may include a digital camera (not shown) or the like for storing and/or transmitting images from within the body 10 to a central processor 200, thereby providing a visual indication of the processes within the body 10. Any of a wide variety of measurements and detections may be made by the present invention, and as such, the detection circuit 120 is selected based on the particular application for which the device 70 is configured. It will also be understood that the detection circuit 120 may be programmable so that it may perform any of a number of various measurements and/or detections. A programmable detection circuit 120 would be able to receive instructions via the circuit 140 which, in the configuration of a programmable detection circuit 120 would be in the form of a transceiver, not merely a transmitter. The memory circuit 130 of the device 70 may be used to store measurement data, programming data or any other data required for operation of the device 70. It is not necessary for the device 70 to include a memory circuit 130.

The elements 120, 130, 140 of the device 70 are encapsulated 150 in a suitable inert material that will not be rejected by the body 10, or otherwise cause any significant complications. For example, the encapsulating material may be titanium, or a material surrounded by an inert surgical grade plastic, or the like. Other materials may be suitable at the molecular level of the devices 70. The encapsulant may also be such that after a predetermined time, the encapsulant will be allowed to dissolve, thereby permitting the devices 70 to be passed from the body 10 after use is complete.

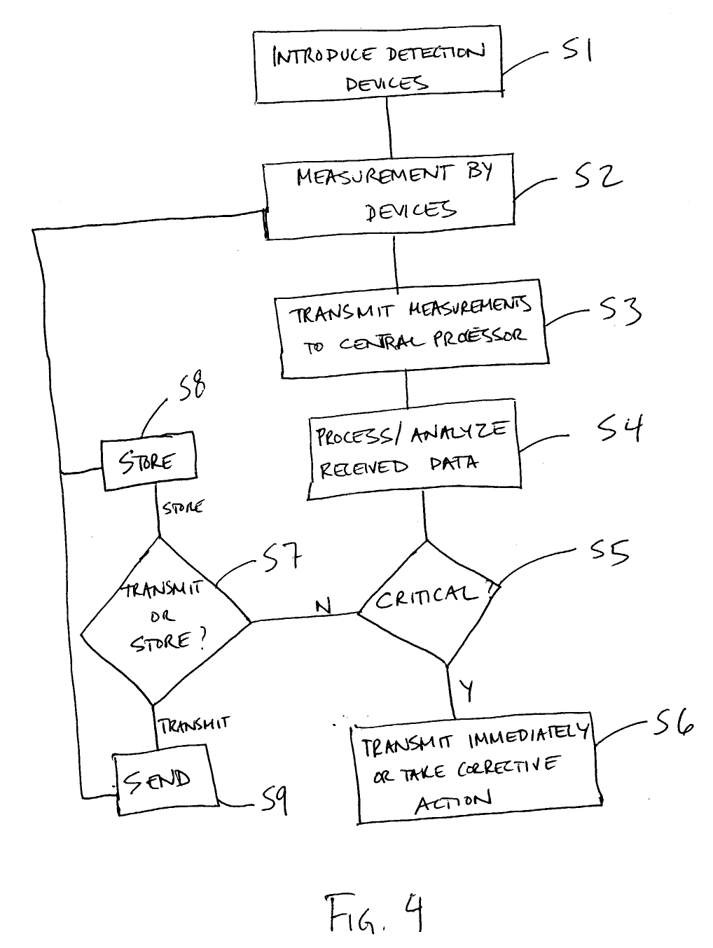

Operation of an exemplary system according to an embodiment of the present invention will be described herein with reference to the flowchart of FIG. 4. It will be understood that the exemplary system contemplates both real-time transmission of measurements to the central processor 200, and periodic transmission of data to the central processor 200. Likewise, it will be understood that the central processor 200 may be configured to transmit its data in real-time to an appropriate authority, or may do so periodically or may change its transmission timing based on the detection of a significant physiological event.

It may also be advantageous to include groups of devices targeted at a particular type of measurement. These groups of devices may be configured to transmit their measurements on separate channels of the communications medium, there being a separate channel for each group of devices. In this exemplary arrangement, the central processor 200 will automatically be informed of the type of measurement being received by virtue of the channel on which such data is received. Alternatively, each individual device 70 may be configured to transmit on its own unique and separate channel. Moreover, each device 70 also preferably has an identification number associated therewith and the data transmitted by each device 70 is preferably tagged with such identification data.

The devices 70 are introduced S1 into the venous system 20 of the body 10, for example, by injection of a liquid suspension containing a plurality of the devices 70. The devices 70 then circulate through the body 10 via the venous system 20 taking various measurements S2 for which they are configured. Examples of the types of measurements being made include, but are not limited to, ultrasonic measurement of effective blood vessel diameter, measurement of fluid pressure (including blood pressure), blood sugar levels, cholesterol levels, heart rate, heart rhythms, detection of the presence of tumors, elevated white blood cell count, and the like. These types of measurements are well known and are readily implemented in the devices 70 of the present invention.

The measurements taken by the devices 70 are then transmitted S3 either in real-time or periodically to the central processor 200. As described above, there may be more than one central processor 200 used to facilitate the determination of the location of a particular device to more accurately determine where a certain condition is occurring. For example, a device 70 may detect a narrowing of the diameter of a major blood vessel, such as, for example, an artery. Using well known triangulation and/or time of transmission techniques, having multiple different receivers will enable the system to determine where the device 70 sending a particular measurement may be, to thereby facilitate more efficient treatment.

The central processor 200 then processes the data S4 received from the devices 70. A determination S5 may then be made as to whether any of the data gives rise to a critical situation requiring immediate attention. For example, detecting an arrhythmia and acting to defibrillate. Otherwise, a determination S7 is made whether to store S8 or transmit S9 data to a predetermined authority for further analysis or diagnosis. It will be understood that the central processor 200 may receive a download command from a predetermined authority requesting that all stored data be immediately downloaded to the authority. This will enable convenient monitoring by the appropriate authority for ongoing diagnoses.

As set forth above, the operation of the central processor 200 may be altered by reprogramming signals sent via the communications medium 80 by an authorized authority.

It will also be understood that the present invention is equally applicable to monitoring conditions of mechanical devices. For example, the devices 70 may be introduced into an engine or turbines in a power plant to detect defects in various areas that may result in future catastrophic failure, expensive repairs or inefficient operation. For example, the devices 70 may be introduced into the hydraulic system of a Space Shuttle to detect microscopic defects or leaks in the tubing that could possibly result in failure of the hydraulic system. Early detection of such microscopic defects may serve to avert catastrophic failure in flight or may be used during design and testing to improve existing systems, and the like.

While the invention has been described in conjunction with exemplary embodiments thereof, it is evident that many alternatives, modifications and variations will be apparent to those skilled in the art. Accordingly, the exemplary embodiments of the invention, as set forth herein, are intended to be illustrative, not limiting. Various changes may be made without departing from the true spirit and full scope of the invention, as defined in the following claims.

What is claimed is:

1. A device for intravenous measurement of physiological data, comprising:
a detection circuit for acquiring physiological data; and
a communication circuit operatively coupled to said detection circuit, said communication circuit comprising a transmitter for transmitting said physiological data acquired by said detection circuit,
wherein said device is encapsulated in an inert material and said encapsulated device having a size to enable said encapsulated device to travel freely within a venous system of a host.

2. The device according to claim 1, further comprising a memory for storing at least some of said physiological data acquired by said detection circuit.

3. The device according to claim 2, wherein said memory stored program data relating to the operation of the device.

4. The device according to claim 1, wherein the communication circuit comprises a transceiver.

5. The device according to claim 1, wherein the detection circuit is capable of acquiring or detecting at least one of the group comprising: effective blood vessel diameter, fluid pressure, blood sugar level, cholesterol level, heart rate, heart rhythm, presence of a tumor, white blood cell count and visual image data.

6. The device according to claim 1, wherein the device is a micro-level or nano-level device.

7. A method for monitoring, recording and reporting physiological data, comprising:
introducing an intravenous detection device into a venous system of a host;
measuring, via said detection device, at least one physiological data;
transmitting said measured physiological data from said detection circuit to a central processor; and
transmitting said measured physiological data from said central processor to an authorized recipient of said data.

8. The method according to claim 7, further comprising storing said physiological data in a memory.

9. The method of claim 7, wherein said step of transmitting to an authorized recipient is performed in real-time

10. The method of claim 7, wherein said step of transmitting to an authorized recipient is performed periodically.

11. The method of claim 7, further comprising:
determining, by said central processor, if a measurement received from a detection device requires immediate action; and
performing an immediate action based on said determining step.

12. The method according to claim 11, wherein said immediate action comprises defibrillating said host.

13. The method according to claim 7, wherein said authorized recipient is capable of transmitting a download command to said central processor to cause immediate transmission of data to said authorized recipient.

14. The method according to claim 13, wherein said data being downloaded is at least one of stored data and data being currently received.

15. The method according to claim 7, wherein said data physiological data acquired by said detection device is at least one of the group comprising: effective blood vessel diameter, fluid pressure, blood sugar level, cholesterol level, heart rate, heart rhythm, presence of a tumor, white blood cell count and visual image data.

16. The method of claim 7, wherein said step of introducing the detection device comprises:
suspending at least one said detection device in a liquid solution; and
injecting said solution with said suspended detection device into the venous system of the host.

17. A system for monitoring, recording and reporting physiological data, comprising:
an intravenous detection device for measuring physiological data of a host in which said detection device is introduced; and
a central processor for receiving said physiological data from said intravenous detection device via a communication medium.

18. The system according to claim 17, wherein said detection device comprises:
a transducer for measuring or acquiring physiological data; and
a communication circuit for transmitting said physiological data to said central processor.

19. The system according to claim 18, wherein said detection device further comprises a memory for storing said measured or acquired physiological data.

20. The system according to claim 17, wherein said detection device is encapsulated in an inert material, said encapsulated device having a size to enable said encapsulated device to travel freely within a venous system of a host.

21. The system according to claim 18, wherein said communication circuit comprises a transceiver.

22. The system according to claim 17, wherein said central processor comprises:
a processor circuit;
a memory; and
a transmitter,
wherein said processor circuit is capable of analyzing said physiological data and determining further action to be taken, including transmitting said data to an authorized recipient.

23. The system according to claim 22, wherein said memory stored physiological data received by said central processor.

24. The system according to claim 17, further comprising multiple central processors.

25. The system according to claim 17, wherein the physiological data acquired by said detection device is at least one of the group comprising: effective blood vessel diameter, fluid pressure, blood sugar level, cholesterol level, heart rate, heart rhythm, presence of a tumor, white blood cell count and visual image data.
